(19) United States
(12) Patent Application Publication
(10) Pub. No.: US 2022/0401449 A1
(43) Pub. Date: Dec. 22, 2022

(54) COMPOSITIONS PROVIDING SLOW RELEASE OF CAFFEINE AND BEVERAGES COMPRISING THE SAME

(71) Applicant: PepsiCo, Inc., Purchase, NY (US)

(72) Inventors: Yu WANG, New York, NY (US); DaSom NO, White Plains, NY (US); Yuan FANG, Cortlandt Manor, NY (US); Nelson TRUSLER, Cortlandt Manor, NY (US); Damian BROWNE, Old Greenwich, CT (US); Xin ZHAO, Shanghai (CN); Meng TIAN, Luoyang (CN); Shiyi ZHANG, Shanghai (CN); George MEYER, White Plains, NY (US); Rohil BHATNAGAR, White Plains, NY (US)

(21) Appl. No.: 17/843,205

(22) Filed: Jun. 17, 2022

(30) Foreign Application Priority Data
Jun. 17, 2021 (CN) 202110670303.4

(51) Int. Cl. A61K 31/522; A61K 47/54; A61K 47/69; A23L 2/385; A23L 2/68

(52) U.S. Cl. CPC A61K 31/522; A61K 47/542; A61K 47/6905; A23L 2/385; A23L 2/68; A23V 2002/00

(57) ABSTRACT

The present disclosure relates to colloids comprising caffeine-tannic acid complexes which slow the release of the caffeine. Also described are methods of preparing the colloids and beverages comprising colloids comprising caffeine-tannic acid complexes.

### FIELD

The present disclosure relates to colloids comprising complexes comprising caffeine and tannic acid. The colloids slow the release of caffeine when used in a beverage. Also described are methods of preparing colloids comprising caffeine-tannic acid complexes.

### BACKGROUND

Caffeine is one of the most widely used pharmacologically active compounds. Previous studies have determined that it is absorbed by the gastrointestinal tract, with peak concentrations occurring between 15 and 120 minutes following ingestion (“Caffeine use in sports, pharmacokinetics in man, and cellular mechanisms of action,” Crit Rev Food Sci Nutr. 2005; 45(7-8); 535-62). Most beverages, either through their express design or through the natural action, provide all of their caffeine all at once. This can cause a “caffeine high,” which can manifest as jitteriness, and can be followed by a “caffeine crash.” To date, there have not been any systems developed that effectively attenuate caffeine release to reduce or ameliorate these potential negative effects.

### SUMMARY

In a first aspect, the present disclosure provides a colloid comprising: a complex comprising caffeine and tannic acid; at least one surfactant; and water. In a first embodiment of the first aspect, the weight/weight ratio of caffeine to tannic acid in the complex is from about 10:1 to about 1:10. In a second embodiment of the first aspect, the weight/weight ratio of caffeine to tannic acid in the complex is from about 5:1 to about 1:5. In a third embodiment of the first aspect, the weight/weight ratio of caffeine to tannic acid in the complex is from about 4:1 to about 1:4.

In a fourth embodiment of the first aspect, the colloid comprises from about 0.01 wt % to about 45 wt % caffeine. In a fifth embodiment of the first aspect, the colloid comprises about 0.5 wt % caffeine. In a sixth embodiment of the first aspect, the colloid comprises from about 0.01 wt % to about 45 wt % tannic acid. In a seventh embodiment, the colloid comprises about 1.5% tannic acid.

In an eighth embodiment of the first aspect, the colloid comprises from about 0.01 wt % to about 55 wt % of a complex comprising from about a 10:1 to about a 1:10 wt/wt ratio of caffeine to tannic acid. In a ninth embodiment of the first aspect, the colloid comprises from about 1 wt % to about 2 wt % of a complex comprising from about a 3:1 to about a 1:3 wt/wt ratio of caffeine to tannic acid.

In a tenth embodiment of the first aspect, the colloid comprises from about 0.01 wt % to about 40 wt % of at least one surfactant. In an eleventh embodiment of the aspect, the colloid comprises about 1 wt % surfactant. In a twelfth embodiment of the first aspect, the surfactant is selected from the group consisting from gum arabic, Quillaia extract, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, and sucrose. In a thirteenth embodiment of the first aspect, the surfactant is polysorbate 80.

In a second aspect, the preset disclosure provides a process for preparing a colloid as described above, the process comprising: adding tannic acid to a solution of caffeine in water; adding at least one surfactant; and agitating the resulting mixture.

In a third aspect, the present disclosure provides a beverage comprising a colloid as described above.

In a fourth aspect, the present disclosure provides a beverage syrup comprising a colloid as described above.

### BRIEF DESCRIPTION OF THE DRAWINGS

The present disclosure is illustrated by way of example, and not limited by, the accompanying figures.

FIG. 1A depicts the FT IR spectrum of caffeine.

FIG. 1B depicts the FT IR spectrum of tannic acid.

FIG. 2A depicts the FT IR spectrum of a 1:1 mixture of caffeine and tannic acid.

FIG. 2B depicts the FT IR spectrum of a complex comprising a 1:1 wt/wt ratio of tannic acid:caffeine.

FIG. 3 depicts the FT IR spectra of a 1:3 mixture of caffeine and tannic acid, a complex comprising a 1:3 wt/wt ratio of caffeine:tannic acid as prepared through an aqueous precipitation method, and a complex comprising a 1:3 wt/wt ratio of caffeine:tannic acid that has been spray dried.

FIG. 4A depicts the change in the amount of free caffeine over time when a colloid comprising 1% polysorbate 80 and a 3:1 wt/wt ratio of tannic acid to caffeine was diluted in deionized water and analyzed by centrifugation.

FIG. 4B depicts the change in the amount of encapsulated caffeine over time when a colloid comprising 1% polysorbate 80 and a 3:1 wt/wt ratio of tannic acid to caffeine was diluted in deionized water and analyzed by centrifugation using 1K molecular weight cutoff.

FIG. 5A depicts the change in the amount of free caffeine over time when a colloid comprising 1% polysorbate 80 and a 3:1 wt/wt ratio of tannic acid to caffeine was diluted in pH 3 buffer and analyzed by centrifugation.

FIG. 5B depicts the change in the amount of encapsulated caffeine over time when a colloid comprising 1% polysorbate 80 and a 3:1 wt/wt ratio of tannic acid to caffeine was diluted in pH 3 buffer and analyzed by centrifugation.

FIG. 6 depicts the change in the amount of free caffeine over time when a colloid comprising 1% polysorbate 60 and a 2:1 wt/wt ratio of tannic acid to caffeine was diluted in deionized water and analyzed by centrifugation using 1K molecular weight cutoff.

### DETAILED DESCRIPTION

### Definitions

The singular forms “a,” “an,” and “the” include plural referents unless the context clearly dictates otherwise.

As used herein, the term “or” is a logical disjunction (i.e., and/or) and does not indicate an exclusive disjunction unless expressly indicated as such with the terms “either,” “unless,” “alternatively,” and words of similar effect.

As used herein, the term “about” refers to ±10% of the noted value, unless otherwise specified, and unless the upper bound of the range would exceed 100% of the composition, in which case the upper limit of the range is limited to 99.9%. Thus, and by way of example only, a composition including about 10 weight percent of a given ingredient could have from 9 to 11 weight percent of the compound. Similarly, a composition including about 95 weight percent of a given ingredient could have from 85.5 to 99.9 weight percent of the ingredient in the composition.

As used herein, “beverage” refers to edible formulations suitable for drinking. Examples of beverages include, but are not limited to, water, soft drinks, fountain beverages, frozen ready-to-drink beverages, coffee beverages, tea beverages, sport drinks, juices, dairy beverages, and alcoholic beverages. Beverages can be carbonated or noncarbonated and can be clear, i.e. transparent, semi-transparent, or opaque. As used herein, “fountain beverages” refer to beverages prepared by combining a beverage syrup and water, which can be optionally carbonated, at or just prior to the point of consumption.

As used herein, the term “colloid” refers to a mixture in which microscopically dispersed insoluble particles of one substance, such as a caffeine-tannic acid complex, are suspended throughout another substance, such as an aqueous phase (i.e. water). In some embodiments, the colloids can comprise one or more surfactants. In some embodiments, the colloids do not comprise an oil phase.

As used herein, the term “surfactant” refers to an agent that lowers the surface or interfacial tension between two liquids, between a gas and a liquid, or between a liquid and a solid. Examples of suitable surfactants include, but are not limited to, gum arabic, Quillaia extract, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof. Additional examples of surfactants will be apparent to those skilled in the art of food or beverage formulations, given the benefit of this disclosure.

All percentages provided in this specification are percentages by weight, unless specifically indicated otherwise.

### Caffeine-Tannic Acid Complex

In certain embodiments, the present disclosure provides a complex comprising caffeine and tannic acid. In some aspects, the complex can be present in a colloid, which, when added to a beverage, can provide a sustained release of caffeine. In some embodiments, the weight/weight ratio of caffeine to tannic acid in the complex can be from about 10:1 to about 1:10. In some embodiments, the weight/weight ratio of caffeine to tannic acid in the complex can be from about 9:1 to about 1:9. In some embodiments, the weight/weight ratio of caffeine to tannic acid in the complex can be from about 8:1 to about 1:8. In some embodiments, the weight/weight ratio of caffeine to tannic acid in the complex can be from about 7:1 to about 1:7. In some embodiments, the weight/weight ratio of caffeine to tannic acid in the complex can be from about 6:1 to about 1:6. In certain embodiments, the weight/weight ratio of caffeine to tannic acid in the complex can be from about 5:1 to about 1:5. In some embodiments, the weight/weight ratio of caffeine to tannic acid in the complex can be from about 4:1 to about 1:4. In some embodiments, the weight/weight ratio of caffeine to tannic acid in the complex can be from about 3:1 to about 1:3. In some embodiments, the weight/weight ratio of caffeine to tannic acid in the complex can be from about 2:1 to about 1:2. In certain embodiments, the weight/weight ratio of caffeine to tannic acid in the complex can be about 1:1. In certain embodiments, the weight/weight ratio of caffeine to tannic acid in the complex can be about 1:2. In certain embodiments, the weight/weight ratio of caffeine to tannic acid in the complex can be about 1:3.

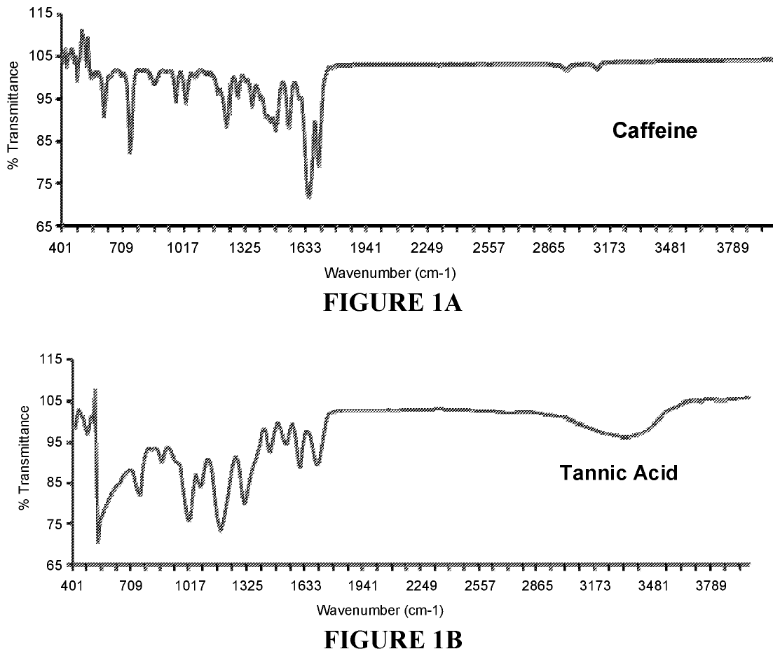

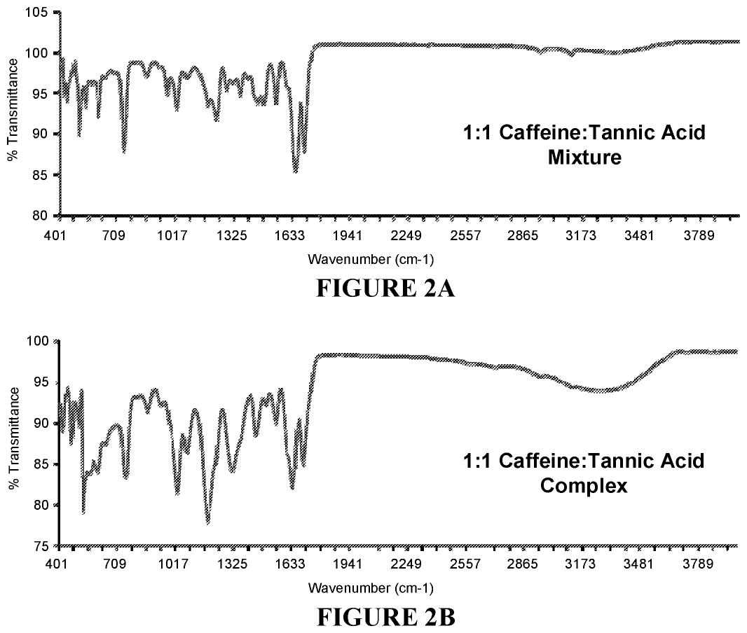

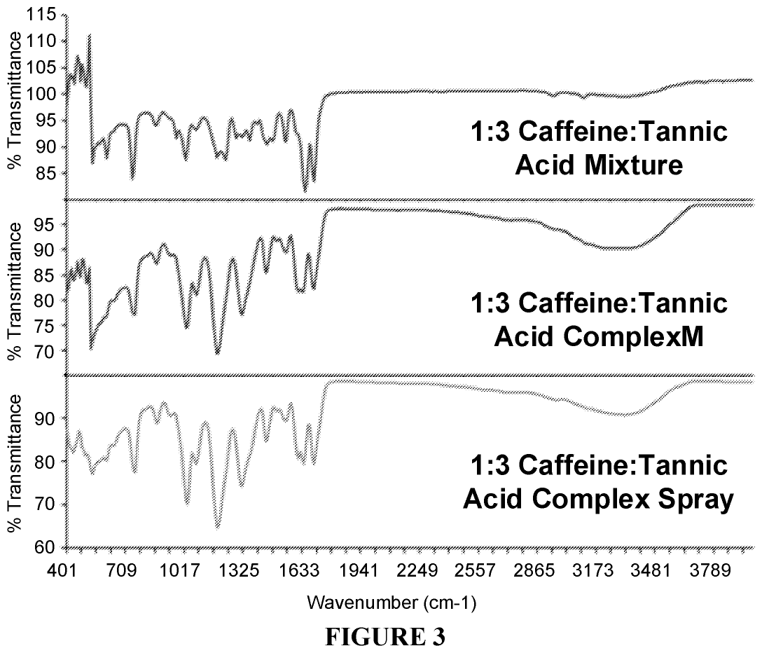

Without wishing to be bound by a particular theory, it believed that under the conditions described herein, tannic acid forms hydrogen bonds with the caffeine molecules to provide a stable complex which, when used in a beverage, slows the release of caffeine from the beverage. Formation of a caffeine-tannic acid complex is supported by spectroscopic analysis, such as FT-IR data. Samples of a 1:1 wt/wt caffeine:tannic acid and a 1:3 wt/wt caffeine:tannic acid complex were analyzed by FT IR and compared to non-complexed mixtures of the reagents in the same ratios. As shown in FIGS. 1A, 1B, 2A, 2B, and 3, the FT IR spectra of the complexes differ from those of the mixed reagents, further confirming that the caffeine and tannic acid are associated within the complex.

### Colloids Comprising the Complex

In certain aspects, the present disclosure provides colloids comprising a caffeine-tannic acid complex, at least one surfactant, and an aqueous phase. In some embodiments, the colloid does not contain an oil phase. In certain embodiments, the weight percent of caffeine present in the colloid can be from about 0.01% to about 45%. In some embodiments, the weight percent of caffeine present in the colloid can be from about 0.02% to about 43%, from about 0.03% to about 41%, from about 0.04% to about 40%, from about 0.05% to about 35%, from about 0.06% to about 30%, from about 0.07% to about 25%, from about 0.08% to about 20%, from about 0.09% to about 15%, from about 0.1% to about 10%, from about 0.1% to about 9%, from about 0.1% to about 8%, from about 0.1% to about 7%, from about 0.1% to about 6%, from about 0.15% to about 5%, from about 0.2% to about 4%, from about 0.3% to about 3%, from about 0.4% to about 2%, or from about 0.5% to about 1%. In certain embodiments, the weight percent of caffeine present in the colloid can be about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 20%, about 25%, about 30%, about 35%, about 40%, or about 45%.

In certain embodiments, the weight percent of tannic acid present in the colloid can be from about 0.01% to about 45%. In some embodiments, the weight percent of tannic acid present in the colloid can be from about 0.02% to about 43%, from about 0.03% to about 41%, from about 0.04% to about 40%, from about 0.05% to about 35%, from about 0.06% to about 30%, from about 0.07% to about 25%, from about 0.08% to about 20%, from about 0.09% to about 15%, from about 0.1% to about 10%, from about 0.1% to about 9%, from about 0.1% to about 8%, from about 0.1% to about 7%, from about 0.1% to about 6%, from about 0.15% to about 5%, from about 0.2% to about 4%, from about 0.3% to about 3%, from about 0.4% to about 2%, or from about 0.5% to about 1%. In certain embodiments, the weight percent of tannic acid present in the colloid can be about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 20%, about 25%, about 30%, about 35%, about 40%, or about 45%.

In certain embodiments, the particle size of the caffeine-tannic acid complex in the colloid prior can be from about 50 nm to about 6000 nm, from about 60 nm to about 5000 nm, from about 70 nm to about 4000 nm, from about 80 nm to about 3000 nm, from about 90 nm to about 2000 nm, from about 100 nm to about 1000 nm, from about 100 nm to about 900 nm, from about 100 nm to about 800 nm, from about 100 nm to about 700 nm, from about 100 nm to about 600 nm, from about 100 nm to about 500 nm, from about 100 nm to about 400 nm, from about 100 nm to about 300 nm, or from about 100 nm to about 200 nm. In some embodiments, the particle size of the complex can be about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1500 nm, about 2000 nm, about 2500 nm, about 3000 nm, about 3500 nm, about 4000 nm, about 4500 nm, about 5000 nm, about 5500 nm, or about 6000 nm.

In some embodiments, the colloid can comprise from about 0.01 wt % to about 40 wt % of the at least one surfactant. In some embodiments, the colloid can comprise from about 0.02 wt % to about 35 wt %, from about 0.03 wt % to about 30 wt %, from about 0.04 wt % to about 25 wt %, from about 0.05 wt % to about 20 wt %, from about 0.06 wt % to about 15 wt %, from about 0.07 wt % to about 14 wt %, from about 0.08 wt % to about 13 wt %, from about 0.09 wt % to about 12 wt %, from about 0.1 wt % to about 11 wt %, from about 0.15 wt % to about 10 wt %, from about 0.2 wt % to about 9 wt %, from about 0.3 wt % to about 8 wt %, from about 0.4 wt % to about 7 wt %, or from about 0.5 wt % to about 6 wt % of the at least one surfactant. In some embodiments, the colloid can comprise about 0.01 wt %, about 0.02 wt %, about 0.03 wt %, about 0.04 wt %, about 0.05 wt %, about 0.06 wt %, about 0.07 wt %, about 0.08 wt, about 0.09 wt %, about 0.1 wt %, about 0.2%, about 0.3 wt %, about 0.4 wt %, about 0.5 wt %, about 0.6 wt %, about 0.7 wt %, about 0.8 wt %, about 0.9 wt %, about 1%, about 1.1 wt %, about 1.2 wt %, about 1.3 wt %, about 1.4 wt %, about 1.5 wt %, about 1.6 wt %, about 1.7 wt %, or about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3.0 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4.0 wt %, about 4.1 wt %, about 4.2 wt %, about 4.3 wt %, about 4.4 wt %, about 4.5 wt %, about 4.6 wt %, about 4.7 wt %, about 4.8 wt %, about 4.9 wt %, about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, about 10.0 wt %, about 11 wt %, about 12 wt %, about 13 wt %, about 14 wt %, about 15 wt %, about 20 wt %, about 25 wt %, about 30 wt %, about 35 wt %, or about 40 wt % of the at least one surfactant.

In certain embodiments, the at least one surfactant can be selected from the group consisting of gum arabic, modified starch, whey protein isolate, palmitic acid, pectin, Quillaia extract, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof. In some embodiments, the can be gum arabic. In some embodiments, surfactant can be polysorbate 80.

As noted earlier, the colloids described herein can also comprise an aqueous phase. In certain embodiments, the colloid can comprise from about 45 wt % to about 99.98 wt % of an aqueous phase. In some embodiments, the colloids can comprise from about 50 wt % to about 99.98 wt %, from about 55 wt % to about 99.98 wt %, from about 60 wt % to about 99.98 wt %, from about 65 wt % to about 99.98 wt %, from about 70 wt % to about 99.98 wt %, from about 75 wt % to about 99.98 wt %, from about 80 wt % to about 99.98 wt %, from about 85 wt % to about 99.98 wt %, from about 90 wt % to about 99.98 wt %, from about 91 wt % to about 99.5% of the aqueous phase, from about 92 wt % to about 99 wt % of the aqueous phase from about 95 wt % to about 99 wt % of the aqueous phase, or about 99 wt % of the aqueous phase.

In certain embodiments, the colloid can comprise from about 0.01 wt % to about 55 wt % of a complex comprising from about a 10:1 to about a 1:10 wt/wt ratio of caffeine to tannic acid, from about 0.1 wt % to about 10 wt % of at least one surfactant, and water. In certain embodiments, the colloid can comprise from about 0.05 wt % to about 55 wt % of a complex comprising from about a 10:1 to about a 1:10 wt/wt ratio of caffeine to tannic acid, from about 0.1 wt % to about 10 wt % of at least one surfactant, and water. In some embodiments, the colloid can comprise about 0.1 wt % to about 55 wt % of a complex comprising from about a 10:1 to about a 1:10 wt/wt ratio of caffeine to tannic acid, about 0.1 wt % to about 10 wt % of at least one surfactant selected from the group consisting of gum arabic, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof, and water.

In certain embodiments, the colloid can comprise about 0.5 wt % to about 35 wt % of a complex comprising from about a 7:1 to about a 1:7 wt/wt ratio of caffeine to tannic acid and comprises from about 0.3 wt % to about 8 wt % of at least one surfactant, and water. In some colloids, the colloid can comprise a) about 0.5 wt % to about 35 wt % of a complex comprising from about a 7:1 to about a 1:7 wt/wt ratio of caffeine to tannic acid and comprises about 0.3 wt % to about 8 wt % of at least one surfactant selected from the group consisting of gum arabic, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof, and water.

In certain embodiments, the colloid can comprise about 1 wt % to about 15 wt % of a complex comprising from about a 5:1 to about a 1:5 wt/wt ratio of caffeine to tannic acid, from about 0.5 wt % to about 6 wt % of at least one surfactant, and water. In some colloids, the colloid can comprise a) about 1 wt % to about 15 wt % of a complex comprising from about a 5:1 to about a 1:5 wt/wt ratio of caffeine to tannic acid and comprises about 0.5 wt % to about 6 wt % of at least one surfactant selected from the group consisting of gum arabic, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof, and water.

In certain embodiments, the colloid can comprise about 2 wt % of a complex comprising about a 1:3 wt/wt ratio of caffeine to tannic acid, about 1 wt % of at least one surfactant, and water. In some embodiments, the colloid can comprise about 2 wt % of a complex comprising about a 1:3 wt/wt ratio of caffeine to tannic acid, about 1 wt % of at least one surfactant selected from the group consisting of gum arabic, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof, and water. In some embodiments, the at least one surfactant can be polysorbate 80.

In certain embodiments, the colloid can comprise about 1 wt % to about 2 wt % of a complex comprising about a 1:1 wt/wt ratio of caffeine to tannic acid, about 1 wt % of at least one surfactant, and water. In some embodiments, the colloid can comprise about 1 wt % to about 2 wt % of a complex comprising about a 1:1 wt/wt ratio of caffeine to tannic acid, about 1 wt % of at least one surfactant selected from the group consisting of gum arabic, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, sucrose ester, and combinations thereof, and water. In some embodiments, the at least one surfactant can be polysorbate 80.

### Processes

In certain embodiments, and in general, the colloid can be prepared by adding tannic acid to an aqueous caffeine solution while mixing under low shear conditions to form a precipitate, and then adding the surfactant to form the colloid. In some embodiments, the aqueous caffeine solution can be prepared by adding caffeine to water at room temperature. In some embodiments, the caffeine can be added to water that has been heated, such as to about 30° C., to about 40° C., to about 45° C., to about 50° C., to about 55° C., or to about 60° C. prior to adding the caffeine. In some embodiments, the aqueous caffeine solution contains a detectable amount of caffeine up to caffeine's saturation concentration in water at a given temperature (for example about 16 mg/ml at room temperature up to about 200 mg/ml at 80° C.). In other embodiments, the amount of caffeine added to the water to form the aqueous caffeine solution prior to forming the complex or the colloid, can exceed caffeine's solubility in the water at a given temperature (i.e. the aqueous caffeine solution is saturated and further comprise caffeine solids).

In other embodiments, the colloid can be prepared by adding tannic acid (neat or as an aqueous solution) to an aqueous mixture of caffeine and surfactant while mixing under standard high shear conditions. In some embodiments, the aqueous caffeine solution can be prepared by adding caffeine to water at room temperature. In some embodiments, the caffeine can be added to water that has been heated, such as to about 30° C., to about 40° C., to about 45° C., to about 50° C., to about 55° C., or to about 60° C. prior to adding the caffeine. In some embodiments, the aqueous caffeine solution contains a detectable amount of caffeine up to caffeine's saturation concentration in water at a given temperature (for example about 16 mg/ml at room temperature up to about 200 mg/ml at 80° C.). In other embodiments, the amount of caffeine added to the water to form the aqueous caffeine solution prior to adding the surfactant, can exceed caffeine's solubility in the water at a given temperature (i.e. the aqueous caffeine solution is saturated and further comprise caffeine solids).

### Beverages

In certain embodiments, the present disclosure provides beverages comprising the colloids described herein. In some embodiments, the colloids can be used to prepare beverages. In some embodiments, the colloids can be added to a beverage syrup which can be diluted to provide a beverage. In some embodiments, the colloids can be added to a pre-prepared beverage.

In some embodiments, the concentration of caffeine in the beverage after addition of the colloid, or after dilution of an appropriate beverage syrup, can be from about 50 ppm to about 900 ppm. In some embodiments, the concentration of caffeine in the beverage can be from about from about 75 ppm to about 850 ppm, from about 100 ppm to about 800 ppm, from about 125 ppm to about 750 ppm, from about 150 ppm to about 700 ppm, from about 175 ppm to about 650 ppm, from about 200 ppm to about 550 ppm of caffeine, or from about 225 ppm to about 500 ppm. In some embodiments, the concentration of caffeine in the beverage can be about 50 ppm, about 75 ppm, about 100 ppm, about 125 ppm, about 150 ppm, about 175 ppm, about 200 ppm, about 225 ppm, about 250 ppm, about 275 ppm, about 300 ppm, about 325 ppm, about 350 ppm, about 375 ppm, about 400 ppm, about 425 ppm, about 450 ppm, about 475 ppm, about 500 ppm, about 525 ppm, about 550 ppm, about 575 ppm, about 600 ppm, about 625 ppm, about 650 ppm, about 675 ppm, about 700 ppm, about 725 ppm, about 750 ppm, about 775 ppm, or about 800 ppm. In some embodiments, the concentration of caffeine in the beverage can be about 400 ppm. If the beverage is prepared from a syrup comprising the colloid, the concentration of caffeine in the syrup will be a multiple of the concentration identified above. For example, if the syrup is converted into a beverage using a 5+1 throw, a technique commonly used in the art, the concentration of caffeine in the syrup before dilution will be 6 times the concentration of caffeine in the resulting beverage. It is within the skill of the ordinarily skilled artisan to determine the concentration of caffeine in the syrup, and, concurrently, the concentration of the colloid in a syrup, when provided with a beverage having a particular caffeine and/or colloid concentration.

In certain embodiments, a portion of the caffeine remains bound to the tannic acid for about 30 minutes to about 180 minutes after the beverage is consumed. In some embodiments, a portion of the caffeine remains bound to the tannic acid for about 45 minutes to about 150 minutes, or about 60 minutes to about 120 minutes after the beverage is consumed. In some embodiments, a portion of the caffeine remains bound to the tannic acid for about 30 minutes, about 40 minutes, about 50 minutes, about 60 minutes, about 70 minutes, about 80 minutes, about 90 minutes, about 100 minutes, about 120 minutes, about 130 minutes, about 140 minutes, about 150 minutes, about 160 minutes, about 170 minutes, or about 180 minutes after the beverage is consumed.

In certain embodiments, the percentage of caffeine bound to the tannic acid 90 minutes after consuming the beverage can be from about 50% to about 99%. In some embodiments, the percentage of caffeine bound to the tannic acid can be from about 65% to about 95%, from about 70% to about 90%, or from about 75% to about 85%. In some embodiments, about 50%, about 55%, about 60%, about 65%, about 70%, about 75%, about 80%, about 85%, about 90%, about 95%, or about 99% of the caffeine is bound to the tannic acid 90 minutes after the beverage is consumed.

In some embodiments, the colloid can be added to a pre-prepared beverage using a “dosing cap.” As used herein, the term “dosing cap” refers to a dispensing container closure system comprising a moisture and oxygen restricted chamber that stores a precise amount of a formula (e.g., a colloid as described herein). The formula can be mixed with the container's contents (i.e., a beverage as described herein) at the time of consumption by removing the closure system from the container. Examples of dosing caps can be found in, for example, U.S. Pat. Nos. 10,266,322; 9,365,335; and 7,032,745; each of which is incorporated by reference in its entirety.

In some embodiments, the dosing cap can comprise an amount of colloid sufficient to achieve a caffeine concentration in the beverage of from about 50 ppm to about 900 ppm of caffeine when the colloid stored in the dosing cap is released and combined with the pre-prepared beverage. In other embodiments, the dosing cap comprises an amount of colloid sufficient to achieve a caffeine concentration of from about 75 ppm to about 850 ppm, from about 100 ppm to about 800 ppm, from about 125 ppm to about 750 ppm, from about 150 ppm to about 700 ppm, from about 175 ppm to about 650 ppm, from about 200 ppm to about 550 ppm of caffeine, or from about 225 ppm to about 500 ppm when combined with the pre-prepared beverage. In some embodiments, the dosing cap can comprise an amount of colloid sufficient to achieve a caffeine concentration of about 50 ppm, about 75 ppm, about 100 ppm, about 125 ppm, about 150 ppm, about 175 ppm, about 200 ppm, about 225 ppm, about 250 ppm, about 275 ppm, about 300 ppm, about 325 ppm, about 350 ppm, about 375 ppm, about 400 ppm, about 425 ppm, about 450 ppm, about 475 ppm, about 500 ppm, about 525 ppm, about 550 ppm, about 575 ppm, about 600 ppm, about 625 ppm, about 650 ppm, about 675 ppm, about 700 ppm, about 725 ppm, about 750 ppm, about 775 ppm, or about 800 ppm when combined with a beverage. In some embodiments, the dosing cap can comprise an amount of colloid sufficient to achieve a caffeine concentration of about 400 ppm in the pre-prepared beverage when combined with the beverage.

In certain embodiments, the beverage can be a carbonated or non-carbonated soft drink, a fountain beverage, a frozen ready-to-drink beverage, a coffee, a tea or other brewed beverage, a dairy beverage, a flavored water, an enhanced water, a juice such as a fruit juice (including diluted and ready-to-drink concentrated juices), a fruit juice-flavored drink, a sport drink, a smoothie, a functionally enhanced beverage such as an energy drink, or an alcoholic beverage. In particular embodiments, the beverage can be a carbonated soft drink. In some embodiments, the beverage can be a caffeinated water.

In certain embodiments, the beverages can comprise one or more sweeteners. Sweeteners of beverage embodiments include caloric carbohydrate sweeteners, natural high-potency sweeteners, synthetic high-potency sweeteners, other sweeteners, and combinations thereof.

Examples of suitable caloric carbohydrate sweeteners include sucrose, fructose, glucose, erythritol, maltitol, lactitol, sorbitol, mannitol, xylitol, D-tagatose, trehalose, galactose, rhamnose, cyclodextrin (e.g., α-cyclodextrin, β-cyclodextrin, and k-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, nigerooligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), lactulose, melibiose, raffinose, rhamnose, ribose, isomerized liquid sugars such as high fructose corn/starch syrup (e.g., HFCS55, HFCS42, or HFCS90), coupling sugars, soybean oligosaccharides, and glucose syrup.

As used herein, the phrase “natural high-potency sweetener,” includes, but is not limited to, rebaudioside A, rebaudioside B, rebaudioside C (dulcoside B), rebaudioside D, rebaudioside E, rebaudioside F, rebaudioside H, rebaudioside I, rebaudioside J, rebaudioside K, rebaudioside L, rebaudioside M, rebaudioside N, rebaudioside O, rebaudioside R, rebaudioside S, rebaudioside T, rebaudioside U, rebaudioside V, dulcoside A, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and cyclocarioside I.

Natural high potency sweeteners also include modified natural high potency sweeteners. Modified natural high potency sweeteners include natural high potency sweeteners which have been altered naturally. For example, a modified natural high potency sweeteners include, but are not limited to, natural high potency sweeteners that have been fermented, contacted with enzyme, derivatized, or substituted. In one embodiment, at least one modified natural high potency sweeteners can be used in combination with at least one natural high potency sweeteners. In another embodiment, at least one modified natural high potency sweeteners can be used without a natural high potency sweeteners. Modified natural high potency sweeteners can be substituted for a natural high potency sweeteners or can be used in combination with natural high potency sweeteners for any of the embodiments described herein.

As used herein, the phrase “synthetic sweetener” refers to any composition that is not found in nature and is a high potency sweetener. Non-limiting examples of synthetic sweeteners suitable for embodiments of this invention include, but are not limited to, sucralose, acesulfame potassium, aspartame, alitame, saccharin, neohesperidin dihydrochalcone, cyclamate, neotame, N-[3-(3-hydroxy-4-methoxyphenyl)propyl]-L-a-aspartyl]-L-phenylalanine 1-methyl ester, N-[3-(3-hydroxy-4-methoxyphenyl)-3-methylbutyl]-L-a-aspartyl]-Lphenylalanine 1-methyl ester, N-[3-(3-methoxy-4-hydroxyphenyl)propyl]L-a-aspartyl]-L-phenylalanine 1-methyl ester, salts thereof (as appropriate), and combinations thereof.

Carbon dioxide can be used to provide effervescence to certain embodiments of the beverages disclosed here. Any of the techniques and carbonating equipment known in the art for carbonating beverages can be employed. Carbon dioxide can enhance beverage taste and appearance and cam aid in safeguarding beverage purity by inhibiting and/or destroying objectionable bacteria. In certain embodiments, for example, the beverage can have a CO₂ level up to about 4.0 volumes carbon dioxide. Other embodiments can have, for example, from about 0.5 to about 5.0 volumes of carbon dioxide. As used herein, one volume of carbon dioxide refers to the amount of carbon dioxide absorbed by a given quantity of a given liquid, such as water, at 60° F. (16° C.) and one atmospheric pressure. A volume of gas occupies the same space as does the liquid by which it is dissolved. The carbon dioxide content can be selected by those skilled in the art based on the desired level of effervescence and the impact of the carbon dioxide on the taste or mouthfeel of the beverage.

In some embodiments, the beverage can further include additional ingredients, including, generally, any of those typically found in beverage compositions. Examples of such additional ingredients include, but are not limited to, caramel and other coloring agents or dyes, foaming or antifoaming agents, gums, emulsifiers, tea solids, cloud components, and mineral and non-mineral nutritional supplements. Examples of non-mineral nutritional supplement ingredients are known to those of ordinary skill in the art and include, for example, antioxidants and vitamins, including Vitamins A, D, E (tocopherol), C (ascorbic acid), B (thiamine), B2 (riboflavin), B6, B12, K, niacin, folic acid, biotin, and combinations thereof. The optional non-mineral nutritional supplements are typically present in amounts generally accepted under good manufacturing practices. Exemplary amounts can be between about 1% and about 100% Recommended Daily Value (RDV), where such RDVs are established. In certain exemplary embodiments the non-mineral nutritional supplement ingredient(s) can be present in an amount of from about 5% to about 20% RDV, where established.

In certain embodiments, the beverages can also include one or more preservatives. Solutions with a pH below 4 and especially those below 3 typically are “micro-stable,” i.e., they resist growth of microorganisms, and so are suitable for longer term storage prior to consumption without the need for further preservatives. However, an additional preservative system can be used if desired. As used here, the terms “preservative system” or “preservatives” include all suitable preservatives approved for use in beverage compositions, including, without limitation, such known chemical preservatives as benzoates, such as sodium, calcium, and potassium benzoate, sorbates, such as sodium, calcium, and potassium sorbate, citrates, such as sodium citrate and potassium citrate, polyphosphates, such as sodium hexametaphosphate (SHMP), and mixtures thereof, and antioxidants such as ascorbic acid, EDTA, BHA, BHT, TBHQ, dehydroacetic acid, dimethyldicarbonate, ethoxyquin, heptylparaben, and combinations thereof. Preservatives can be used in amounts not exceeding mandated maximum levels under applicable laws and regulations. In some embodiments, the beverages can include potassium sorbate.

In certain embodiments, the beverages can include an antioxidant selected from the group consisting of rutin, quercetin, flavonones, flavones, dihydroflavonols, flavonols, flavandiols, leucoanthocyanidins, flavonol glycosides, flavonone glycosides, isoflavonoids, and neoflavonoids. In particular, the flavonoids may be, but not limited to, quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, oolong tea polymerized polyphenol, anthocyanin, heptamethoxyflavone, daidzin, daidzein, biochaminn A, prunetin, genistin, glycitein, glycitin, genistein, 6,7,4′ trihydroxy isoflavone, morin, apigenin, vitexin, balcalein, apiin, cupressuflavone, datiscetin, diosmetin, fisetin, galangin, gossypetin, geraldol, hinokiflavone, primuletin, pratol, luteolin, myricetin, orientin, robinetin, quercetagetin, and hydroxy-4-flavone.

The beverages described herein can also optionally include one or more suitable food grade acids. Exemplary acids are water soluble organic acids and their salts and include, but are not limited to, phosphoric acid, sorbic acid, ascorbic acid, benzoic acid, citric acid, tartaric acid, propionic acid, butyric acid, acetic acid, succinic acid, glutaric acid, maleic acid, malic acid, valeric acid, caproic acid, malonic acid, aconitic acid, potassium sorbate, sodium benzoate, sodium citrate, amino acids, and combinations of any of them. In particular embodiments, the beverages include malic acid and/or phosphoric acid.

The embodiments described herein are further detailed with reference to the examples shown below. These examples are provided for the purpose of illustration only and the embodiments described herein should in no way be construed as being limited to these examples. Rather, the embodiments should be construed to encompass any and all variations which become evident as a result of the teachings provided herein.

### EXAMPLES

### Example 1A: General Procedure for Preparing Colloid Containing Caffeine-Tannic Acid Complex

Caffeine (1 g) was added to water (99 g) and agitated until fully dissolved. The caffeine solution was placed under 600 rpm shear, and treated with a solution of tannic acid (either 2 g tannic acid in 98 g water or 3 g tannic acid in 97 g of water, depending on the complex being formed). One weight equivalent of a surfactant described herein (relative to total caffeine-tannic acid complex weight) was added either neat or predissolved in water, depending on the surfactant used, and the resulting mixture was stirred to provide the colloid.

### Example 1B: Alternative Procedure for Preparing Colloid Containing Caffeine-Tannic Acid Complex

Caffeine was added to water and agitated until fully dissolved. The caffeine solution was placed under 600 rpm shear, and treated with one weight equivalent (relative to total caffeine-tannic acid complex weight) of a surfactant as described herein. The resulting solution was treated with tannic acid (either 2 g tannic acid in 98 g water or 3 g tannic acid in 97 g water, depending on the complex being formed) and stirred to provide the colloid.

Table 1 illustrates colloids prepared by the methods described above.

TABLE 1

| | Tannic | | | |
|---|---|---|---|---|
| | Acid/ | | | |
| | Caffeine | | | Complex |
| | Ratio | Surfactant | Preparation | Particle |
| Example | (wt/wt) | Type | Method | Size |
| 1a-1 | 3:1 | Polysorbate 80 | Example 1A | 160 nm |
| 1a-2 | 2:1 | Polysorbate 80 | Example 1A | 142 nm |
| 1a-3 | 3:1 | Gum Arabic | Example 1A | 749 nm |
| 1a-4 | 3:1 | Polysorbate 60 | Example 1A | 166 nm |
| 1b-1 | 3:1 | Polysorbate 80 | Example 1B | 571 nm |
| 1b-2 | 3:1 | Polysorbate 60 | Example 1B | 737 nm |

### Example 2: Procedure for Determining the Release Rate of Caffeine from Colloids Using Centrifugation

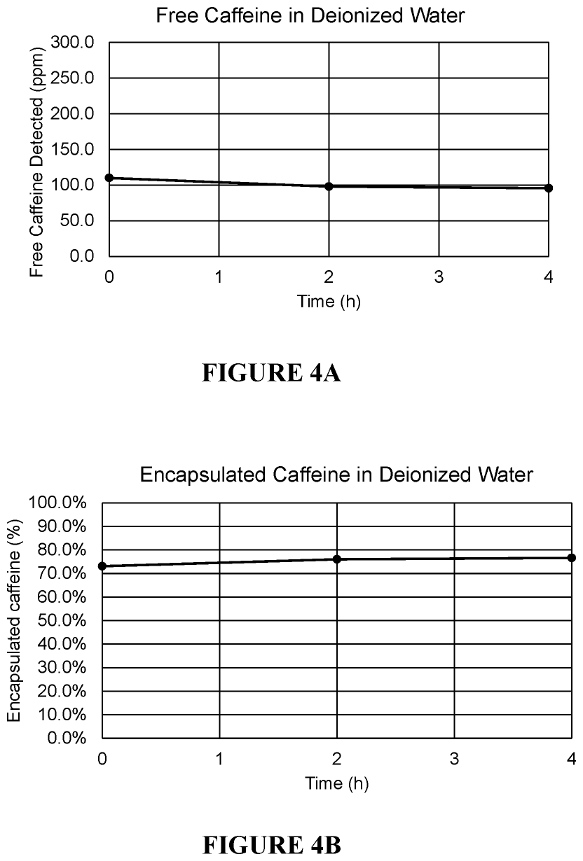

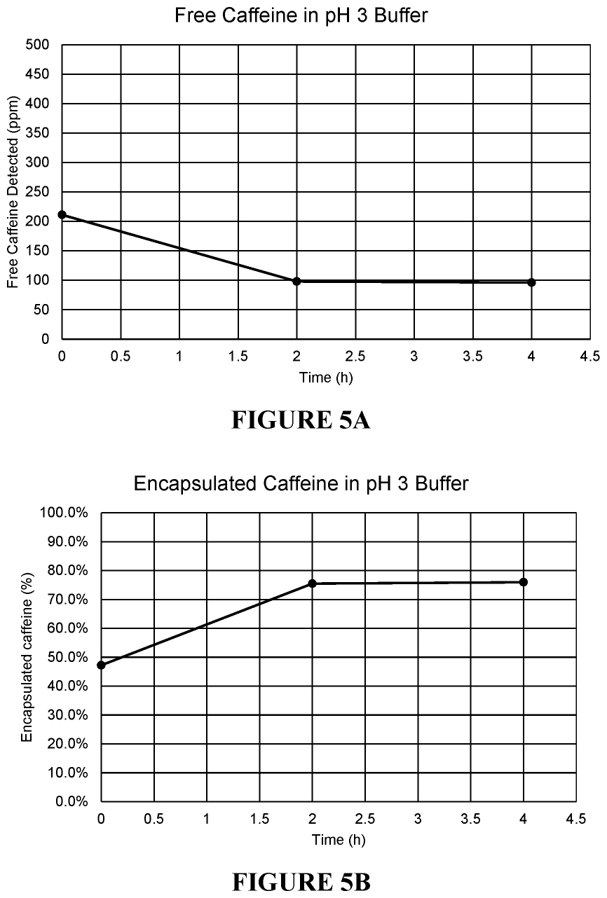

For a typical experiment, 10 g of colloid (as prepared in Example 1) was mixed with either 250 g of deionized water or 250 g of pH 3 buffer as prepared in Table 2 (to simulate a soft drink) to form a solution with a target caffeine concentration of 400 ppm. At each sampling time, an aliquot was removed and loaded into a centrifuge tube with 1K molecular weight cut off. The tube was centrifuged at 5,300 rpm for 30 min in order to filter the sample through the cut off membranes. The amount of free and encapsulated caffeine as determined over time for colloid Example 1a-1 are shown in Tables 3 and 4 and FIGS. 4A, 4B, 5A, and 5B. As shown in the tables and figures, the percentage of encapsulated caffeine (or the amount of free caffeine) remains stable over time. These results show that beverages comprising the colloid can retain caffeine in encapsulated form. and could provide a slow release of caffeine after consumption

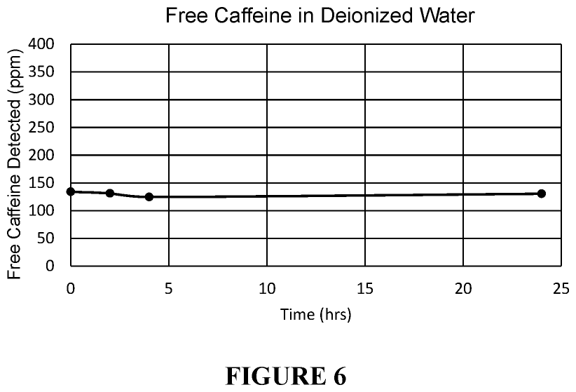

The amount of free caffeine as determined over time for a Example 1a-2 diluted with deionized water to a caffeine concentration of 400 ppm is shown in Table 5 and FIG. 6. As shown in the table and figure, the percentage of free caffeine remained stable over time.

TABLE 2

| | Per Liter Batch |
|---|---|
| Ingredient | 3.0 pH |
| Citric Acid | 1.1300 gms |
| Tri Sodium Citrate | 0.1600 gms |
| Sodium Benzoate | 0.1575 gms |
| Water in L | 1 |
| (QS to volume) | |

TABLE 3

| | Released | |
|---|---|---|
| | “Free” | Remaining |
| | Caffeine in | Encapsulated |
| Time | Deionized | in Deionized |
| (h) | Water | Water |
| 0 | 110.0 | 73.1% |
| 2 | 97.9 | 76.0% |
| 4 | 95.5 | 76.6% |

TABLE 4

| | Released | |
|---|---|---|
| | “Free” | Remaining |
| | Caffeine | Encapsulated |
| Time | in pH 3 | in pH 3 |
| (h) | Buffer (ppm) | Buffer |
| 0 | 211.1 | 47.2% |
| 2 | 98.0 | 75.5% |
| 4 | 96.1 | 76.0% |

TABLE 45

| | Released |
|---|---|
| | “Free” |
| | Caffeine in |
| Time | Deionized |
| (h) | Water (ppm) |
| 0 | 134.1 |
| 2 | 131.2 |
| 4 | 124.6 |
| 24 | 130.4 |

The breadth and scope of the present disclosure should not be limited by any of the above-described exemplary embodiments, but should be defined only in accordance with the following claims and their equivalents.

All patents, patent applications, and other reference noted or referenced in this application are hereby incorporated by reference in their entirety

1. A colloid comprising:
a complex comprising caffeine and tannic acid;
at least one surfactant; and
water.

2. The colloid of claim 1, wherein the weight/weight ratio of caffeine to tannic acid in the complex is from about 10:1 to about 1:10.

3. The colloid of claim 1 wherein the weight/weight ratio of caffeine to tannic acid in the complex is from about 5:1 to about 1:5.

4. The colloid of claim 1, wherein the weight/weight ratio of caffeine to tannic acid in the complex is from about 4:1 to about 1:4.

5. The colloid of claim 1, wherein the colloid comprises from about 0.01 wt % to about 45 wt % caffeine.

6. The colloid of claim 1, wherein the colloid comprises about 0.5 wt % caffeine.

7. The colloid of claim 1, wherein the colloid comprises from about 0.01 wt % to about 45 wt % tannic acid.

8. The colloid of claim 1, wherein the colloid comprises about 1.5% tannic acid.

9. The colloid of claim 1, wherein the colloid comprises from about 0.01 wt % to about 55 wt % of a complex comprising from about a 10:1 to about a 1:10 wt/wt ratio of caffeine to tannic acid.

10. The colloid of claim 1, wherein the colloid comprises from about 1 wt % to about 2 wt % of a complex comprising from about a 3:1 to about a 1:3 wt/wt ratio of caffeine to tannic acid.

11. The colloid of claim 1, wherein the colloid comprises from about 0.01 wt % to about 40 wt % of at least one surfactant.

12. The colloid of claim 1, wherein the colloid comprises about 1 wt % surfactant.

13. The colloid of claim 1, wherein the surfactant is selected from the group consisting from gum arabic, Quillaia extract, modified starch, whey protein isolate, palmitic acid, pectin, sodium caseinate, lecithin, lactoferrin, dioctyl sodium sulfosuccinate, polysorbate 20, polysorbate 40, polysorbate 60, polysorbate 65, polysorbate 80, and sucrose.

14. The colloid of claim 1, wherein the surfactant is polysorbate 80.

15. A process for preparing the colloid of claim 1, the process comprising: adding tannic acid to a solution of caffeine in water; adding at least one surfactant; and agitating the resulting mixture.

16. A beverage comprising the colloid of claim 1.

17. A beverage syrup comprising the colloid of claim 1.
